(19) United States
(12) Patent Application Publication
(10) Pub. No.: US 2026/0207354 A1
(43) Pub. Date: Jul. 23, 2026

(54) TORSION ADAPTER

(71) Applicant: Bulldog Tools, Inc., Lewisburg, OH (US)

(72) Inventors: Matthew Habecker, Zionsville, IN (US); Robert Meyer, Lewisburg, OH (US)

(21) Appl. No.: 19/456,756

(22) Filed: Jan. 22, 2026

Related U.S. Application Data
(60) Provisional application No. 63/748,066, filed on Jan. 22, 2025.

(51) Int. Cl. A61F 2/76; A61F 2/50

(52) U.S. Cl. CPC A61F 2/76; A61F 2002/5073; A61F 2220/0025

(57) ABSTRACT

A torsion adapter for use with prosthetic limbs comprises a receiver portion and a coupler portion. The receiver portion includes a receiver plate, a spring groove, and an aperture offset from a center of the receiver portion. The coupler portion includes a coupler, a spring groove complementary to the receiver portion spring groove, and an aperture offset from a center of the coupler portion. When the receiver portion is coupled to the coupler portion, the spring grooves align to create a spring pocket, which includes a spring. When the receiver portion is coupled to the coupler portion, the apertures align to create an offset axis of rotation such that the coupler portion may rotate about the offset axis from a set position relative to the receiver portion and the spring biases the coupler portion to rotate back to the set position.

### CROSS REFERENCE TO RELATED APPLICATIONS

This application claims the benefit of U.S. Provisional Patent Application Ser. No. 63/748,066, filed Jan. 22, 2025, having the title TORSION ADAPTER, by Habecker et al., the entire disclosure of which is hereby incorporated herein by reference.

### BACKGROUND

Various aspects of the present disclosure relate generally to exoskeletal prosthetic devices and more specifically to a torsion adapter for a exoskeletal prosthetic devices.

An exoskeletal prosthetic device is a prosthetic device that can help replace function of a missing body part, including limbs. Some limb exoskeletal prosthetic devices include a torsion adapter that increases transverse plane compliance of the prosthesis and decreases torque applied to a residual limb associated with the exoskeletal prosthetic device.

### BRIEF SUMMARY

According to aspects of the present disclosure, a torsion adapter for use with prosthetic limbs comprises a receiver portion and a coupler portion. The receiver portion includes a receiver plate, a spring groove; and an aperture offset from a center of the receiver portion. The coupler portion includes a coupler, a spring groove complementary to the receiver portion spring groove, and an aperture offset from a center of the coupler portion. When the receiver portion is coupled to the coupler portion, the spring grooves align to create a spring pocket, which includes a spring. When the receiver portion is coupled to the coupler portion, the apertures align to create an offset axis of rotation such that the coupler portion may rotate about the offset axis from a set position relative to the receiver portion and the spring biases the coupler portion to rotate back to the set position.

According to further aspects of the present disclosure, the spring pockets of the receiver portion and the coupler portion include a linear wall and a curved wall opposite the linear wall. In some of those embodiments, the curved walls are concave.

Moreover, according to aspects of the present disclosure, various embodiments of the torsion adapter include a low-friction plate between the receiver portion and the coupler portion. In some of those embodiments, the low-friction plate includes a cutout that houses a bumper such that when the coupler portion rotates on the offset axis, the bumper dampens terminal rotation of the coupler portion.

Also, according to aspects of the present disclosure, in numerous embodiments, the spring comprises two springs.

According to yet further aspects of the present disclosure, in several embodiments, the coupler portion includes a circumferential latch, and the receiver portion further includes a circumferential latch groove. The circumferential latch couples to the circumferential latch groove to keep the torsion adapter together. In various embodiments, the circumferential latch is on the receiver portion, and the circumferential latch groove is on the coupler portion.

### BRIEF DESCRIPTION OF THE SEVERAL VIEWS OF THE DRAWINGS

FIG. 1A is an exploded view of a drawing of a torsion adapter, according to various aspects of the present disclosure;

FIG. 1B is the exploded view of FIG. 1, where internal portions of the torsion adapter are shown via transparency, according to various aspects of the present disclosure;

FIG. 2 is a view of a drawing of a receiver portion of the torsion adapter of FIG. 1, according to various aspects of the present disclosure; and

FIG. 3 is a view of a drawing of a coupler portion of the torsion adapter of FIG. 1, according to various aspects of the present disclosure.

### DETAILED DESCRIPTION

According to aspects of the present disclosure, a torsion adapter for use with prosthetic limbs includes an axis that is offset from an axis of a residual limb that the prosthetic limb. To achieve the offset axis, an aperture is set offset from a center of two portions of the torsion adapter: a coupler portion and a receiver portion. Further, a curved spring pocket allows for an internal spring to bias the coupler portion to a set position. The offset axis allows for a more comfortable experience for a user. For example, the offset axis helps provide an optimized mechanical advantage for the user's center of mass to rotate through space in a transverse plane.

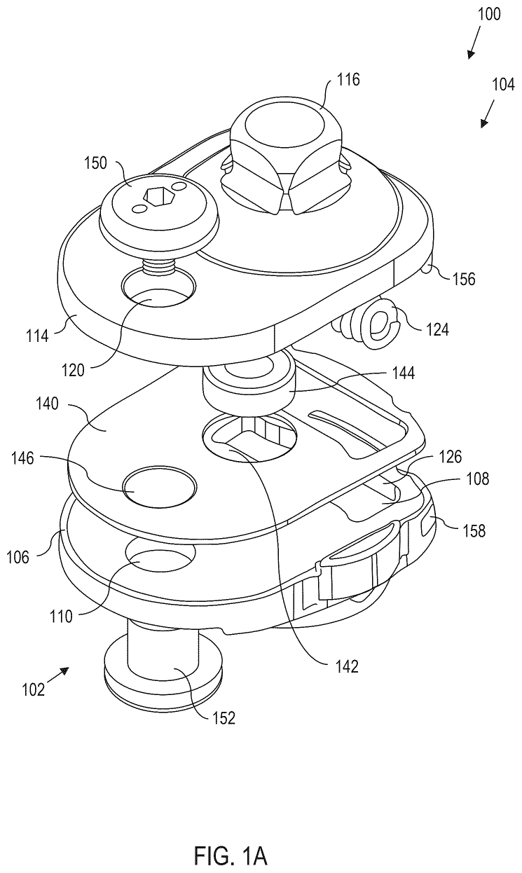

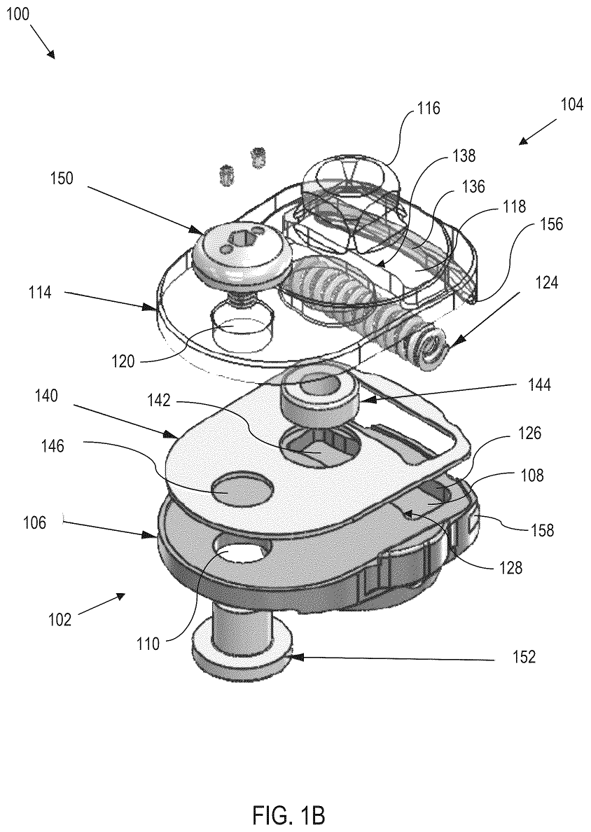

Turning now to the figures, and in particular FIGS. 1A-B, a torsion adapter 100 is shown. In FIG. 1A, the torsion adapter 100 is shown opaquely, while in FIG. 1B, the torsion adapter 100 is transparent to illustrate some of the inner workings of the torsion adapter 100. The torsion adapter 100 includes a receiver portion 102 and a coupler portion 104. The receiver portion 102 includes a receiver plate 106, a spring groove 108, and an aperture 110 that is offset from a center of the receiver portion 102. The coupler portion 104 includes a coupler plate 114 including a coupler 116 for attaching to a prosthetic limb, a spring groove 118 complementary to the spring groove 108 of the receiver portion 102, and an aperture 120 that is offset from a center of the coupler portion 104.

When the receiver portion 102 is coupled to the receiver portion 104, the two spring grooves 108, 118 combine together to form a spring pocket, and a spring 124 is located within the spring pocket. Some embodiments include more than one spring 124 in parallel within the spring groove. In various embodiments, the spring groove 108 of the receiver portion 102 includes a linear wall 126 and a curved wall 128 opposite the linear wall 126, and the spring groove 118 of the coupler portion 104 includes a linear wall 136 and a curved wall 138 opposite the linear wall 136. In some embodiments, the curved walls 128, 138 are concave.

Numerous embodiments of the torsion adapter 100 include a low-friction plate 140 between the receiver portion 102 and the coupler portion 104 that provides a low-friction rotation of the coupler portion 104 relative to the receiver portion 102. The center of rotation of the torsion adapter 100 is a center point of the offset apertures 110, 120 (i.e., an offset axis). Further, the low-friction plate 140 includes an aperture 142 for a bumper 144 that provides a dampening of the rotation of the torsion adapter, so there is not an immediate stop when rotating to a limit of the torsion adapter. Moreover, the low-friction plate includes an aperture that aligns with the apertures 110, 120 of the receiver portion 102 and the coupler portion 104.

The concave walls 128, 138 allow the spring 124 to curve around the wall and follow the rotation of the torsion adapter and then bias the coupler portion back to a non-rotated position when no force is applied to the receiver portion 102 or the coupler portion 104.

An aperture 146 aligns with apertures 110 and 120 to create a passage for the receiver portion 102 couples to the coupler portion 104 via a bolt 150 and nut 152, which creates the offset axis around which the coupler portion rotates. Further, in many embodiments, the coupler portion 104 includes a circumferential latch 156 that extends from a circumference of the coupler portion 104 (based on the offset axis), and the receiver portion 102 includes a corresponding circumferential latch groove 158. Thus, when the receiver portion 102 couples to the coupler portion 104, the latch 156 secures the coupler portion 104 to the receiver portion (along with the bolt 150 and nut 152) and also allows the coupler portion to rotate with respect to the receiver portion while limiting transverse movement.

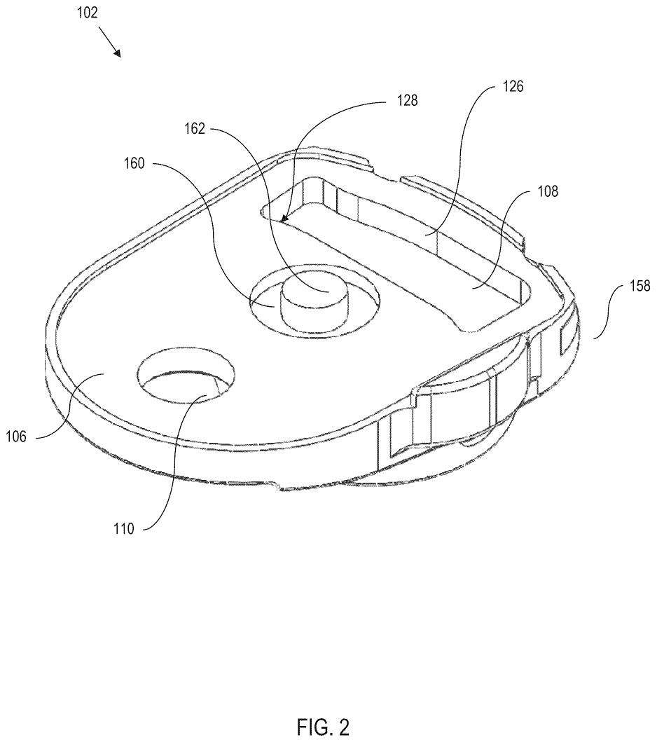

Turning now to FIG. 2, a drawing of the receiver portion 102 is shown. As discussed above, the receiver portion 102 includes a receiver plate 106, a spring groove 108, and an aperture 110. The spring groove 108 includes a linear wall 126 and a curved wall 128. Moreover, the receiver portion 102 includes a circumferential latch groove 158. Further, the receiver portion includes a bumper groove 160 with a post 162 for securing the bumper (144, FIG. 1).

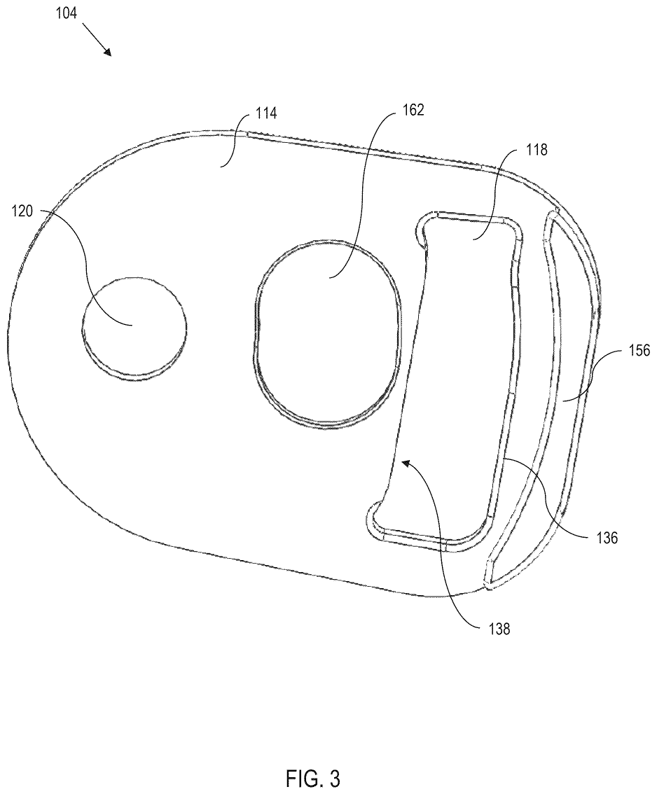

Similarly, turning to FIG. 3, the coupler portion 104 includes the coupler plate 114, the coupler spring groove 118, and the coupler aperture 120. The spring groove 128 includes a flat wall 136 and a curved wall 138. Moreover, the coupler portion 104 includes the circumferential latch 156. Further, the coupler portion 104 includes a non-circular bumper groove 162 that allows for rotation of the coupler portion 104 with respect to the receiver portion 102 with respect to the offset axis, where the rotation is limited by a size of the bumper groove 162 and a size of the bumper (144, FIG. 1).

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. A torsion adapter for use with prosthetic limbs, the torsion adapter comprising:
a receiver portion comprising:
a receiver plate;
a spring groove; and
an aperture offset from a center of the receiver portion;
a coupler portion comprising:
a coupler plate including a coupler for attaching a prosthetic limb;
a spring groove complementary to the receiver portion spring groove; and
an aperture offset from a center of the coupler portion;
wherein:
when the receiver portion is coupled to the coupler portion, the spring groove of the receiver portion and the spring groove of the coupler portion create a spring pocket;
a spring occupies the spring pocket;
when the receiver portion is coupled to the coupler portion, the aperture of the receiver portion aligns with the aperture of the coupler portion to create an offset axis of rotation such that the coupler portion may rotate about the offset axis from a set position relative to the receiver portion; and
the spring biases the coupler portion to rotate back to the set position.

2. The torsion adapter of claim 1, wherein:
the spring groove of the receiver portion includes:
a linear wall; and
a curved wall opposite the linear wall; and
the spring groove of the coupler portion includes:
a linear wall; and
a curved wall opposite the linear wall.

3. The torsion adapter of claim 2, wherein:
the curved wall of the receiver portion is concave; and
the curved wall of the coupler portion is concave.

4. The torsion adapter of claim 1 further comprising a low-friction plate between receiver portion and the coupler portion.

5. The torsion adapter of claim 4, wherein the low-friction plate includes a cutout that houses a bumper such that when the coupler portion rotates on the offset axis, the bumper dampens terminal rotation of the coupler portion.

6. The torsion adapter of claim 1, wherein the spring comprises two springs.

7. The torsion adapter of claim 1, wherein:
the coupler portion further includes a circumferential latch; and
the receiver portion further includes a circumferential latch groove;
wherein the latch couples to the latch groove to keep the torsion adapter together.

8. The torsion adapter of claim 1, wherein:
the receiver portion further includes a circumferential latch; and
the coupler portion further includes a circumferential latch groove;
wherein the latch couples to the latch groove to keep the torsion adapter together.
